(19) United States
(12) Patent Application Publication
(10) Pub. No.: US 2008/0167503 A1
(43) Pub. Date: Jul. 10, 2008

(54) PROCESS FOR PRODUCING ETHERIFIED COMPOUNDS FROM ALCOHOLS

(75) Inventors: Yusrabbil Amiyati Binti YUSOF, Bandar Baru Bangi (MY); Rozana Binti Abu Bakar, Bandar Baru Bangi (MY); Ooi Tian Lye, Bandar Baru Bangi (MY); Salmiah Binti Ahmad, Bandar Baru Bangi (MY)

Correspondence Address: LOWE HAUPTMAN HAM & BERNER, LLP, 1700 DIAGONAL ROAD, SUITE 300, ALEXANDRIA, VA, 22314, US

(73) Assignee: MALAYSIAN PALM OIL BOARD, Kajang (MY)

(21) Appl. No.: 11/972,580

(22) Filed: Jan. 10, 2008

(30) Foreign Application Priority Data
Jan. 10, 2007 (MY) PI 2007 0034

(51) Int. Cl. C07C 41/09

(52) U.S. Cl. 568/698

(57) ABSTRACT

A method to produce alkyl ether comprising the step of reacting short chain polyols with monohydric alcohols in the presence of a catalyst under pre-determined temperature and pressure.

### RELATED APPLICATIONS

The present application is based on, and claims priority from, Malaysian Application Number PI 2007 0034, filed Jan. 10, 2007, the disclosure of which is hereby incorporated by reference herein in its entirety.

### FIELD OF INVENTION

The present invention is related to an improved method to produce etherified compounds simply by reacting two different types of alcohol under influence of a catalyst. In more specific, the disclosed method is able to produce alkyl ether by reacting polyols with another monohydric alcohol.

### BACKGROUND OF THE INVENTION

Etherified compounds have been utilized for different application since decades ago and now becoming more interesting owing to its function as fuel additive in automobiles. It has been shown that diesel fuel with etherified compounds like glycerol alkyl ethers produce much lower contaminants after the combustion.

There are several well-known methods now applied in the field for large scale production of etherified compounds such as nucleophilic displacement of alkyl halides by alkoxides; electrophilic addition of alcohols to alkenes and dehydration of alcohols. It is know in the art that dehydration of alcohol, especially those aliphatic alcohols, is apparently not applicable in the aspect of industrial production. Beside of requiring the reaction conducted under drastic condition, the yield of the etherified compounds from the alcohol dehydration is extremely low. Instead, high portion of the dehydrated alcohol forms the unwanted side products, olefin. Such approach is not practical in industry due to extravagance on the substrates. Nevertheless, patent no EP0781751 filed a patent on a manufacturing method of phenol alkyl ether by reacting phenol with alcohol in the presence of a catalysts. The benzyl ring found in the phenol has much higher resistance against oxidation during the process thus it is reasonable to be applied in this European patent as one of the substitute. On the other hand, Japanese patent no 2000103756 claimed a manufacture approach in producing phenol alkyl ether by reacting phenol with low grade fatty alcohol under the influence of niobic acid in gaseous phase. Thus, it is obvious that the low yield of etherified compounds from dehydration of aliphatic alcohol remained unsolved even the abovementioned patents.

### SUMMARY OF THE INVENTION

The present invention aims to provide an improved method as a resort to resolve the low yield of etherified compounds by dehydration of aliphatic alcohol. Particularly, the present invention employs at least two different types of alcohol which favors the production of etherified compounds instead of the by-products, olefin.

Another object of the present invention is directed to disclose a simple and inexpensive way to obtain high portion of etherified compounds by reacting different types of alcohols with the aid of catalysts under suitable condition.

Still another object of the present invention aims to disclose a method to produce etherified compounds by reacting different types of alcohol which has no need to be performed under drastic environment (high temperature and high acidic environment). Under drastic condition, it is known that the functional groups of both substrates and products are likely to be damaged.

At least one of the preceding objects is met, in whole or in part, by the present invention, in which one of the embodiments of the present invention comprises the steps of reacting short chain polyols with monohydric alcohols in the presence of catalysts under elevated temperature.

In order to achieve the optimum yield from the reaction, the ratio of said short chain polyol to said monohydric alcohol is crucial and preferably remained in the range of 1 to 1:1 to 4 in mole of hydroxyl group contained.

In one of the preferred embodiments, the short chain polyol used is preferably C3 to C8 polyol. In fact the monohydric alcohol used is preferably is a tertiary alcohol.

Another important feature of the present invention is the use of catalysts characterized by having strong acidic functional groups to accelerate the rate of the process. Another important factor is determining the end products yield is the temperature of the environment where the reaction performed, in the preferred embodiment the reaction is conducted under the temperature of between 45° C. to 185° C.

One of the embodiments involves further purification steps to obtain the purified etherified ether compounds from unreacted substrates, catalysts and side products comprises the steps of removing the catalysts; and removing unreacted short chain polyol, aliphatic alcohol and by products to obtain the purified alkyl ether. Aiming to achieve high yield the disclosed method may be conducted under high pressure.

Still other objects and advantages of the present invention will become readily apparent to those skilled in the art from the following detailed description, wherein the preferred embodiments of the invention are shown and described, simply by way of illustration of the best mode contemplated of carrying out the invention. As will be realized, the invention is capable of other and different embodiments, and its several details are capable of modifications in various obvious aspects, all without departing from the invention. Accordingly, the drawings and description thereof are to be regarded as illustrative in nature, and not as restrictive.

### DETAIL DESCRIPTION OF THE INVENTION

One skilled in the art will readily appreciate that the present invention is well adapted to carry out the objects and obtain the ends and advantages mentioned, as well as those inherent therein. The embodiment describes herein is not intended as limitations on the scope of the invention.

The present invention is an improved method to produce alkyl ether characterized by the steps of reacting short chain polyols with monohydric alcohols in the presence of catalysts with elevated temperature. The disclosed method is able to produce alkyl ether up to 70% of the short chain polyols used. It is important to be noted that the types of end product obtained, alkyl ether, depends upon the substrate employed in the reaction.

Besides of this, the amount of polyols and monohydric alcohol used is crucial in order to achieve the maximum yield. The amount of monohydric alcohol employed is relative to the hydroxyl groups contained within the short chain polyols used because the reaction to produce the etherified compounds involves the reaction between hydroxyl functional groups. In the most preferred embodiment, the ratio of monohydric alcohol used compare to the polyols is maintained within the range of 1-4:1-1 in mole of hydroxyl group. It should be mentioned that the ratio provided herein in the disclosure is directed to the object of optimally converting the substrates to the etherified compounds; any of the substrates in fact can be employed in excess though it may end up to be converted to unwanted side products and affect the yield percentage of the etherified compounds.

In accordance to the preferable embodiment, the short chain polyols refers to any one or combination of the polyols having carbon number of 3 to 8 as it carbon backbone, namely C1 to C8 polyols; while in the most preferred embodiment glycerol is utilized as the short chain polyols. On the other hand, the monohydric alcohol applied in the present invention is preferably a tertiary alcohol which is crucial in the process for producing the etherified compounds. Tert-butanol is used in the most preferred embodiment.

It is of important to have the catalysts in the reaction to produce the etherified compounds. The employed catalysts do not involve actively in the reaction but rather provide reactive sites for the alcohols to react with at lower energy level, thus directly enhance the rate of the reaction tremendously. In order to enhance the reaction, catalysts used are preferably containing strong acidic functional group. Conventional catalyst applied for alcohols dehydration is directed to strong acids such as sulphuric acid, nitric acid and the like. In the preferred embodiment, insoluble polymeric catalysts are used. These insoluble polymeric catalysts are engineered polymers, preferably ion exchange resin, anchored with strong acidic functional groups which are now commercially available. Representative examples are Amberlyst. Application of the insoluble polymeric catalysts not only renders the purification procedures easier after the reaction, but also makes the reaction more cost effective as these catalysts can be re-used for numerous reactions.

In the conventional alcohols dehydration method, high temperature is one of the primary requirements in order to produce the etherified compounds. Normally, the temperature used for dehydrating the alcohols can up to 250° C. to 350° C. Under such drastic condition, the produce etherified compounds tend to be further oxidized into the unwanted olefin leading to low yield of the etherified compounds in the reaction. However, the disclosed method in the present invention can be conducted in a much lower temperature from 185° C. to as low as 45° C., more preferably within the range of 60° C. to 100° C. By performing the reaction under such low temperature, over-oxidation of the etherified compounds can be avoided. Moreover, the present invention is possible to be conducted in an environment where positive pressure is applied. The positive pressure acts like concentrating the substrates in the reaction thus lead to better yield.

It is patentably important to be noted that the disclosed method success in providing a high yielding method of etherified compounds by reacting alcohols solely is due to electronic nature and physical structure of the polyols and the monohydric alcohols used. It is known in the art that under normal circumstance the etherified compound is at first produced as the primary product in the reaction, but is consumed to form olefin later on by dehydration of the etherified compounds. It is well known in the art that alcohols are themselves nucleophilic thus can react among themselves to produce ethers and water via dehydration reaction, while drastic condition has to be used to accelerate the rate of conversion which in turn lead to the production of olefin. However, the etherified compounds produced in the present invention in fact are obtained with different route of reaction instead of dehydration solely as in the conventional method. Under E1 elimination reaction, a single molecule alcohol by itself can dehydrate to produce olefin or alkenes in accordance mainly to Zaitsev's Rule. Moreover, tertiary alcohol is more susceptible to the elimination reaction as the water molecule can be easily removed by increasing the temperature slightly above room temperature. For example, the tert-butanol employed in the present invention is converted to 2-methyl-propene when the temperature of the reaction increased to 80° C. Subsequently, the polyols used in the reaction, preferably glycerol, are then reacting with the newly formed 2-methyl-propene through electrophilic addition under the aid of catalysts to produce etherified compounds, namely mono- and di-alkyl glycerol ether. Tri-alkyl glycerol is hardly produced due to the physical structure of the glycerol used. Attention now shall be drawn to the fact that primary and secondary monohydric alcohols are applicable in the present invention though higher temperature is required to dehydrate these alcohols that may lead to higher yield of olefin. Using of polyols like glycerol in the reaction is because of its chemical and electronic nature which is more resistant to further oxidation to olefin at the acidic site through alkoxide pathway.

Pursuant to another embodiment of the present invention, additional steps can be taken for purifying the etherified compounds from the unreacted substrates, catalysts used as well as the side products. The purification steps, in short, include the steps of removing the catalysts; and removing unreacted short chain polyol, aliphatic alcohol and by products to obtain the purified alkyl ether. Depend upon the types of the catalysts used; different approaches can be adapted for removing it from the mixture. In respect to the preferred embodiment which utilizes insoluble polymeric catalysts having the acidic functional groups anchored on it as the catalytic site for the reaction, a simple filtration steps may serve the purpose. For solvent type of catalysts like sulfuric acid, it may form the lower partition in the mixture owing to its hydrophilic properties in oppose to other hydrophobic organic compounds and can be drained away from the bottom of the mixture. Still, there are many ways to attain the purpose of removing catalysts; one skilled in the art shall appreciate that any modification in this aspect shall contain within the scope of the invention. Moreover, water produced from the dehydration of the alcohols can be removed by the abovementioned method also. Likewise, the other organic elements such as unreacted substrate and olefin side products can be removed from the etherified compounds according to their differences in boiling temperature. One of the available approaches to separate the other unreacted organic from the etherified compounds is subjecting the mixture to rotatary evaporator to vaporize the unreacted alcohols and side products. Distillation is another practical approach to purify the etherified compounds by separating the compounds in the mixture at different stages of heating. Bearing in mind that the physical and chemical properties of etherified compounds and the side products produced rely on the types of substrate use, thus it is not the intention of the present invention to limit application of other purification procedures exploiting the properties of the produced products.

The following example is intended to further illustrate the invention, without any intent for the invention to be limited to the specific embodiments described therein.

### EXAMPLE 1

Glycerol (92 g or 1 mole) was charged into a 500 mL 3-neck-round bottom flask and then tert-butanol (296 g or 4 mole) was charged in the same flask together with Amberlyst 15 (4.6 g). The mixture was stirred with a magnetic stirrer and was heated.

The temperature rose from ambient to 80° C. in 1 hour and was maintained at 80° C. to 85° C. for 5 hours. The glycerol dissolved in tert-butanol during the heating process and etherification occurred in homogenous phase. Water formed as a by-product during the reaction period. The progress was monitored via TLC.

### EXAMPLE 2

The cooled reaction product for example 1 was filtered to separate the catalyst. Then the filtrate (product) evaporated to remove water and unreacted tert-butanol. The crude glycerol alkyl ether was subjected to GC analysis and the conversion of glycerol to glycerol alkyl ether was about 70%. The product was also subjected to GC-MS analysis. From the GC-MS analysis, two primary components were determined which were monoalkyl glycerol ether and diakyl glycerol ether. Therefore, the product was a mixture of about 60% monoalkyl glycerol ether and 10% dialkyl glycerol ether.

### EXAMPLE 3

The experiment as example 1 was repeated. Other reactions parameters remained the same except the acid catalyst used was Amberlite IR-120 instead of Amberlyst 15. The product obtained was treated as example 2. The GC analysis showed the conversion of glycerol to glycerol alkyl ethers was about 75%. The product was a mixture of 61% monoalkyl glycerol ether and 14% dialkyl glycerol ether.

### EXAMPLE 4

The same experiment was repeated. Other reactions parameters remained the same except the acid catalyst used was Montmorillonite K10. The product obtained was treated as example 2. The conversion of glycerol to glycerol alkyl ethers was about 64%.

### EXAMPLE 5

The same experiment as example 4 was repeated by using p-toluenesulfonic acid as a catalyst. Other reaction parameters remained the same. Water and unreacted tert-butanol were removed from the reaction product by using rotary evaporator. The conversion of glycerol was about 67%.

### EXAMPLE 6

The same experiment as example 5 was repeated. Other reaction parameters remained the same except the sulphuric acid was used as a catalyst. The conversion of glycerol was about 74%.

The present disclosure includes as contained in the appended claims, as well as that of the foregoing description. Although this invention has been described in its preferred form with a degree of particularity, it is understood that the present disclosure of the preferred form has been made only by way of example and that numerous changes in the details of construction and the combination and arrangements of parts may be resorted to without departing from the scope of the invention.

1. A method to produce alkyl ether comprising the step of reacting short chain polyols with monohydric alcohols in the presence of a catalyst.

2. The method according to claim 1, further comprising the step of subjecting the reacting step under predetermined temperature of 45° C. to 185° C.

3. The method according to claim 1, wherein the reacting step is conducted under high pressure.

4. The method according to claim 1, further comprising the purification steps of:
(a) removing the catalyst; and
(b) removing unreacted short chain polyol, monohydric alcohol and other by-products to obtain the purified alkyl ether.

5. The method according to claim 1, wherein the ratio of said short chain polyol to said monohydric alcohol is 1:1-4 in mole.

6. The method according to claim 1, wherein said short chain polyol is C3 to C8 polyol.

7. The method according to claim 1, wherein said short chain polyol is glycerol.

8. The method according to claim 1, wherein said monohydric alcohol is a tertiary alcohol.

9. The method according to claim 1, wherein said monohydric alcohol is tert-butanol.

10. The method according to claim 1, wherein said catalyst has a strong acidic functional group.

11. The method according to claim 1, wherein said catalyst is insoluble polymeric catalyst.

12. The method according to claim 2, wherein said catalyst is removed by filtration.

13. The method according to claim 2, wherein said unreacted short chain polyol, monohydric alcohol and by products are removed by an evaporation process.

14. The method according to claim 2, wherein the reacting step is conducted under high pressure.

15. The method according to claim 2, further comprising the purification steps of:
(a) removing the catalyst; and
(b) removing unreacted short chain polyol, monohydric alcohol and other by-products to obtain the purified alkyl ether.

16. The method according to claim 3, further comprising the purification steps of:
(a) removing the catalyst; and
(b) removing unreacted short chain polyol, monohydric alcohol and other by-products to obtain the purified alkyl ether.

17. The method according to claim 14, further comprising the purification steps of:
(a) removing the catalyst; and
(b) removing unreacted short chain polyol, monohydric alcohol and other by-products to obtain the purified alkyl ether.

18. The method according to claim 2, wherein the ratio of said short chain polyol to said monohydric alcohol is 1:1-4 in mole.

19. The method according to claim 2, wherein said short chain polyol is C3 to C8 polyol.

20. The method according to claim 2, wherein said short chain polyol is glycerol.
